(19) United States
(12) Patent Application Publication
(10) Pub. No.: US 2013/0158242 A1
(43) Pub. Date: Jun. 20, 2013

(54) ALPHA-SELECTIVE GLYCOSYLATION METHOD

(71) Applicants: Kwok-Kong Tony Mong, Hsinchu City (TW); Chin-Sheng Chao, Hsinchu City (TW); Shao-Ru Lu, Hsinchu City (TW); Chih-Yueh Liu, Hsinchu City (TW)

(72) Inventors: Kwok-Kong Tony Mong, Hsinchu City (TW); Chin-Sheng Chao, Hsinchu City (TW); Shao-Ru Lu, Hsinchu City (TW); Chih-Yueh Liu, Hsinchu City (TW)

(73) Assignee: National Chiao Tung University, Hsinchu City (TW)

(21) Appl. No.: 13/525,696

(22) Filed: Jun. 18, 2012

(30) Foreign Application Priority Data
Dec. 16, 2011 (TW) 100146744

(51) Int. Cl. C07H 1/00

(52) U.S. Cl. 536/18.6

(57) ABSTRACT

The present invention provides an α-selective glycosylation method. The α-selective glycosylation method includes performing a reaction of a donor having a saccharide structure and a formamide-containing compound to form a glycosyl imidate compound; and in one pot environment, performing an addition reaction of the glycosyl imidate compound and an acceptor having a hydroxyl group to form an α-glycoside with high α-selectivity. The α-selective glycosylation method is applicable to the large scale production and easy to recover the formamide-containing compound.

### BACKGROUND OF THE INVENTION

1. Field of the Invention

The present invention relates to an α-glycosylation method, and more particularly to, α-glycosylation method with a compound having a saccharide structure.

2. Description of Related Art

Complex carbohydrates including polysaccharides and oligosaccharides are formed by linkages of saccharide building blocks. Various complex carbohydrates are formed from different linked saccharide units, wherein monosaccharide units are linked via glycosidic bonds. A glycosidic bond is formed between the hemiacetal group of a saccharide and the hydroxyl group of some organic compound such as an alcohol. Glycosidic bonds are classified into α- and β-glycosidic bonds based on the configurations. The synthesis of glycosidic bonds is a complicated process, which requires a control on the stereochemistry. There are some methods for controlling the stereochemistry of glycosidic bond formation. For example, in Org. Bioorg. Chem. 2010, 8, 497-510, 1,2-trans α- and β-glycosidic bonds are formed by the use of neighboring group participation concept. However, there is no simple method for forming 1,2-cis α-glycosidic bonds.

Currently, 1,2-cis α-glycosidic bond is formed by optimized conditions such as using specific ethereal solvents, adding nucleophilic additives, using special hydroxyl protecting function, etc. However, most of these methods suffer from a rather narrow scope of application and they are applicable to only few types of saccharide units. On some occasions, the selectivity of glycosylation is moderate.

US Patent Application Publication No. 2006122379 and U.S. Pat. No. 6,388,059 disclose a α-glycosylation method. However, in this method, the glycoside group donor is activated in the presence of the receptor, and the stereo-selectivity of the reaction is just moderate. Further, in U.S. Pat. No. 6,388,059, the thioglycoside donor needs to be oxidized to give a sulfoxide and thus this method is more complicated.

Hence, there is a need to develop a simple α-glycosylation method with high α-selectivity in 1,2-cis α- and 1,2-trans α-glycosidic bond formations.

### SUMMARY OF THE INVENTION

The present invention provides an α-selective glycosylation method. The method includes the steps of: performing a reaction of a donor having a saccharide structure and a formamide-containing compound to form a glycosyl imidate compound; and performing a coupling reaction of the glycosyl imidate compound with an acceptor having a hydroxyl group to form an α-glycoside.

In the present invention, the saccharide structure of the donor is activated in the absence of an acceptor to form an oxacarbenium compound, which then reacts with the formamide-containing compound. The saccharide structure is activated by an activating agent. Preferably, the carbon atom at the first position of the saccharide structure is substituted with a thioacetal, a halo, a phosphate or an acetimidate. In other words, the above thioacetal, halo, phosphate or acetimidate is used as a leaving group.

In the method of the present invention, there is no specific limitation to the formamide-containing compound. Preferably, the formamide-containing compound has the structure of formula (I):

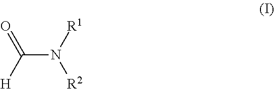

wherein R¹ and R² are independently C₁-C₆alkyl or R¹ and R² are part of a 5-membered or 6-membered-cyclic compound and the cyclic structure can has one or more than one heteroatoms.

The α-selective glycosylation method of the present invention further includes the steps of activating a glycoside donor to react with a formamide-containing compound forming a glycosyl imidate; and performing a coupling reaction of the imidate compound with an acceptor having a hydroxyl group.

In the present invention, the reaction of the formamide-containing compound and the donor having the saccharide structure is performed to form an intermediate, a glycosyl imidate compound, which then reacts in one pot environment with the acceptor having a hydroxyl group in a coupling reaction. Thus, the method of the present invention produces the α-glycoside with high α-selectivity (1,2-cis α-glycoside and 1,2-trans α-glycoside). Therefore, the method of the present invention is suitable for the large scale production, and the formamide-containing compound is easily recovered.

### DETAILED DESCRIPTION OF PREFERRED EMBODIMENTS

The following illustrative embodiments are provided to illustrate the disclosure of the present invention. These and other advantages and effects can be apparently understood by those in the art after reading the disclosure of this specification.

The α-selective glycosylation method includes the steps of: performing a reaction of a donor having a saccharide structure and a formamide-containing compound to form a glycosyl imidate compound; and performing a coupling reaction of the glycosyl imidate compound with an acceptor having a hydroxyl group to form an α-glycoside (containing 1,2-cis α-glycoside and 1,2-trans α-glycoside).

Generally, the mixture of a donor such as a thioglycoside and flame-dried molecular sieve (such as AW300) is suspended in a dried solvent such as CH₂Cl₂, wherein the concentration of the donor in the solution is about 50 to 75 mM. Then, the formamide-containing compound is added into the mixture, and stirred at the room temperature for 10 minutes. The mixture is then stirred at −10° C. for 10 minutes. In the method of the present invention, the saccharide structure of the donor is activated in the absence of an acceptor to form an oxacarbenium compound, which then reacts with the formamide-containing compound. The saccharide structure is activated by an activating agent. Specifically, the carbon at the first position of the saccharide in the donor is substituted, and the substitute is activated by an activating agent, wherein the carbon at the first position of the saccharide is substituted with a thioacetal group, and the activating agent is a halonium ion source. If the carbon at the first position of the saccharide is substituted with a halo, the activating agent is the Ag⁺ or Hg²⁺ ion source. For example, the carbon at the first position 1 of the saccharide is substituted with a thioacetal group, and the halonium ion source is a mixture of N-halosuccinimide and a Lewis acid, wherein the N-halosuccinimide is N-iodosuccinimide or N-bromosuccinimide, and the Lewis acid is triflic acid, trimethylsilyl triflate or silver triflate. For example, the halonium ion source is a mixture of N-iodosuccinimide (NIS) and trimethylsilyl trifluoromethanesulfonate (TMSOTf).

Further, the sulfonate is dimethyl(methylthio)sulfonium triflate, methyl triflate or methylfluorosulfonate, and the tetrafluoroborate is dimethyl(methylthio)sulfonium tetrafluoroborate.

The amount of the activating agent and the reaction conditions may be adjusted according to the reactants. Generally, the amount of the activating agent is 1 equivalent weight of NIS and 1 to 1.5 equivalent weights of TMSOTf, the reaction temperature is −40° C. to 30° C., and the reaction time is 3 to 48 hours.

After the activation, the acceptor having a hydroxyl group is added for the coupling reaction. Upon completion of the coupling reaction, the saturated NaHCO₃ and sodium bisulfite are added and stirred, wherein the blood-red color of the mixture is turned to light yellow. Then, the mixture is dried with magnesium sulfate, filtered and purified by flash chromatography, so as to obtain α-glycoside.

Preferably, the carbon at the first position of the saccharide structure is substituted with a thioacetal, a halo, a phosphate or an acetimidate. In other words, the above thioacetal, halo, phosphate or acetimidate is used as a leaving group.

The substitute may be, but not limited to, a thioacetal such as thiotoluenyl acetal or thiophenyl acetal; an acetimidate such as trichloroacetimidate or N-phenyl trifluoroacetimidate; or a phosphate such as diphenyl phosphate.

In the α-selective glycosylation method of the present invention, the active atom in the saccharide structure of the donor is linked to a protecting group, and the active atom is an oxygen atom or a nitrogen atom. In the method of the present invention, there is no need to use chiral auxiliary protecting groups, and the high α-selectivity is achieved by using common protecting groups.

Further, in the α-selective glycosylation method of the present invention, various donors having saccharide structures may be used. The donor may be mono saccharide or oligosaccharides. Generally, the saccharide structure has at least six carbon atoms, and is a linear or circular structure. The donor having a saccharide structure may be D-galactopyranose, D-glucopyranose, 2-azido-2-deoxy-D-galactopyranose 2-azido-2-deoxy-D-glucopyranose, L-fucopyranose, L-idopyranose, D-mannose, or L-rhamnose.

In the method of the present invention, there is no specific limitation to the formamide-containing compound. Preferably, the formamide-containing compound has the structure of formula (I):

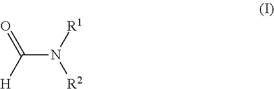

wherein R¹ and R² are independently C₁-C₆alkyl or R¹ and R² are part of a 5-membered or 6-membered cyclic compound that bears one or more than one heteroatom.

The formamide-containing compound may be, but not limited to, N,N-dimethylformamide, N,N-diethylformamide, N,N-diisopropylformamide, N-formyl pyrrolidine, N-formyl piperidine or N-formyl morpholine.

### Embodiment 1

### Synthesis of 2,3-di-O-benzyl-4,6-O-benzylidene-D-galactopyranosyl-(1,6)-1,2:3,4-di-O-isopropylidene-1-α-D-galactopyranose

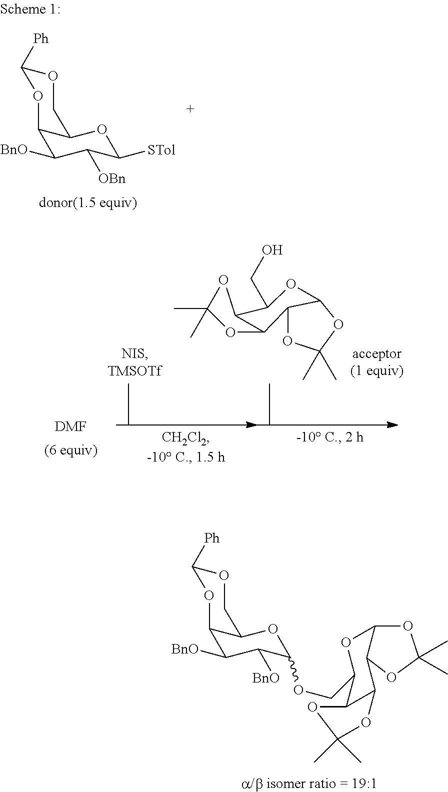

The compound of Embodiment 1 is synthesized according to Scheme 1, wherein Bn is benzyl; Ph is phenyl; and STol is thio-toluene. The mixture of a donor having a thiogalactoside (166.3 mg, 0.3 mmol, according to Z. Zhang, I. R. Ollmann, X.-S. Ye, R. Wischnat, T. Baasov, C.-H. Wong, J. Am. Chem. Soc. 1999, 121, 734-753), N,N-dimethylformamide (DMF, 93 μL, 1.2 mmol) and a flame-dried molecular sieve (for example, AW300) was suspended in dried CH₂Cl₂ (4.0 mL). The mixture was stirred at the room temperature for 10 minutes, and then stirred at −10° C. for 10 minutes. Then, in the absence of an acceptor, NIS (77 mg, 0.34 mmol) and TMSOTf (54 μL, 0.3 mmol) were added. After the activation was performed at −10° C. for 1.5 hours, the galactoside acceptor (52 mg, 0.2 mmol (Alfa Aesar, B24899)) was added, and the addition reaction was performed at −10° C. for 2 hours. After the addition reaction, saturated NaHCO₃ and sodium sulfite were added. Then, the mixture was dried with magnesium sulfate, filtered and analyzed by chromatography (hexane/EtOAc/CH₂Cl₂: 3/1/1), so as to obtain the white glass compound (125 mg, 87%, α/β=19:1).

α-isomer had ¹H NMR (300 MHz, CDCl₃): δ=7.53-7.24 (m, 2H, ArH), 7.51-7.23 (m, 13H, ArH), 5.50 (d, J=6 Hz, 1H, H-1), 5.47 (s, 1H, benzylidene-CH), 5.05 (d, J=3.3 Hz, 1H, H-1′), 4.82 (dd, J=6, 12 Hz, 2H), 4.72 (dd, J=6, 12 Hz, 2H), 4.58 (dd, J=3, 7 Hz, 1H), 4.31-4.27 (m, 2H), 4.20-4.18 (m, 2H), 4.10-3.69 (m, 4H), 3.78-3.69 (m, 3H), 1.52 (s, 3H, CH₃), 1.44 (s, 3H, CH₃), 1.26 (s, 3H, CH₃), 1.24 (s, 3H, CH₃); ¹³C NMR (75 MHz, CDCl₃): δ 138.6, 138.5, 137.7, 128.7, 128.1, 127.9, 127.6, 127.5, 127.40, 127.36, 126.2, 109.1 (isopropylidene-C), 108.4 (isopropyl-C), 100.9 (benzylidene-C), 98.0 (C-1), 96.1 (C-1′), 75.7, 75.3, 74.5, 73.0, 71.8, 70.9, 70.4, 70.3, 69.3, 66.8, 66.4, 62.4, 25.9, 25.8, 24.8, 24.4.

### Embodiment 2

### Synthesis of methyl 2,3,4,6-tetra-O-benzyl-D-glucopyranosyl-α-(1,4)-2,3-O-isopropylidene-1-α-L-rhamnopyranoside

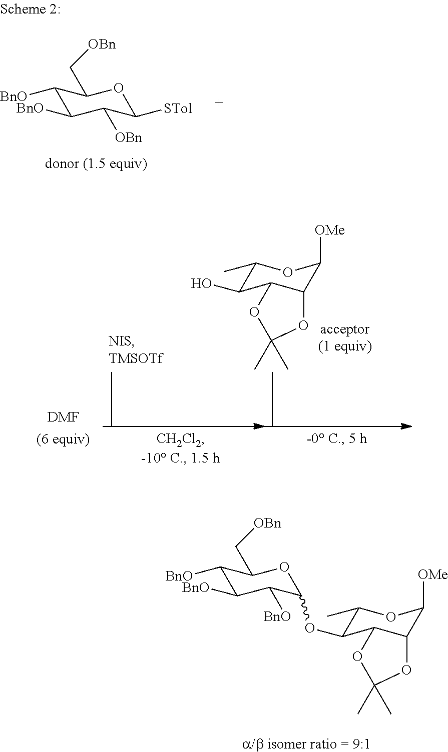

The compound of Embodiment 2 was synthesized according to Scheme 2.

The mixture of a donor having thioglucoside (194.0 mg, 0.3 mmol, according to C.-S. Chao, C.-W. Li, M.-C. Chen, S.-S. Chang, K-K. T. Mong, Chem. Eur. J. 2009, 15, 10972-10982), DMF (93 μL, 1.2 mmol) and DMF (93 μL, 1.2 mmol) and a flame-dried molecular sieve (for example, AW300) was suspended in dried CH₂Cl₂ (4.0 mL). The mixture was stirred at the room temperature for 10 minutes, and then stirred at −10° C. for 10 minutes. Then, in the absence of an acceptor, NIS (77 mg, 0.34 mmol) and TMSOTf (54 μL, 0.3 mmol) were added. After the activation was performed at −10° C. for 1.5 hours, the rhamnoside acceptor (44 mg, 0.2 mmol, according to C.-S. Chao, C.-W. Li, M.-C. Chen, S.-S. Chang, K-K. T. Mong, Chem. Eur. J. 2009, 15, 10972-10982) was added, and the addition reaction was performed at 0° C. for 5 hours. After the addition reaction, saturated NaHCO₃ and sodium sulfite were added. Then, the mixture was dried with magnesium sulfate, filtered and analyzed by chromatography (hexane/EtOAc/CH₂Cl₂: 5/1/1), so as to obtain the creamy white glass compound (111 mg, 75%, α/β=9:1).

α-isomer had ¹H NMR (300 MHz, CDCl₃): δ 7.36-7.23 (m, 18H, ArH), 7.18-7.15 (m, 2H, ArH), 4.98-4.95 (m, 2H), 4.88-4.78 (m, 4H), 4.73-4.60 (m, 2H), 4.52 (d, J=7.5 Hz, 1H), 4.48 (d, J=9 Hz, 1H), 4.12-4.04 (m, 3H), 3.98 (t, J=9.3 Hz, 1H), 3.82-3.70 (m, 3H), 3.65-3.58 (m, 2H), 3.34 (q, J=10.8, 17.1 Hz, 1H), 3.33 (s, 3H, OCH₃), 1.43 (s, 3H, CH₃), 1.31 (d, J=6.3 Hz, 3H, CH₃), 1.25 (s, 3H, CH₃); ¹³C NMR (75 MHz, CDCl₃): δ 138.7, 138.3, 137.9, 137.8, 128.39, 128.38, 128.34, 128.30, 128.24, 127.92, 127.89, 127.8, 127.65, 127.63, 127.5, 108.9 (isopropyl-C), 98.3 (J_{CH}=168 Hz, C-1′), 97.7 (J_{CH}=166 Hz, C-1), 82.2, 80.7, 79.7, 77.74, 77.75, 75.8, 75.5, 75.1, 74.2, 73.5, 70.2 67.9, 64.7, 54.6, 28.1, 26.3, 17.4; HRMS (MALDI-TOF): [M+Na]⁺ C₄₄H₅₂O₁₀Na=63.34527, m/z=763.3478.

### Embodiment 3

### Synthesis of toluenyl 2,3-di-O-benzyl-4,6-di-O-benzylidene-D-glucopyranosyl-α-(1,6)-2,3-di-O-benzoyl-4-O-benzyl-1-thio-α-D-glucopyranoside

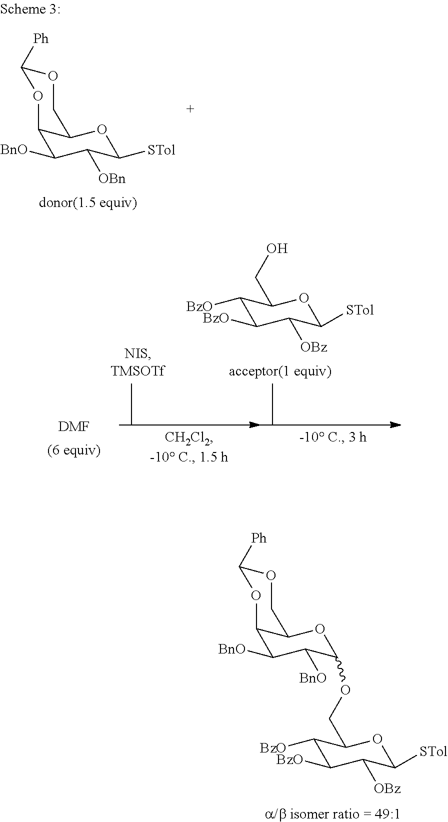

The compound of Embodiment 3 was synthesized according to Scheme 3, wherein Bz is benzoyl.

The mixture of a donor having thiogalactoside (166.3 mg, 0.3 mmol), DMF (93 μL, 1.2 mmol) and DMF (93 μL, 1.2 mmol) and a flame-dried molecular sieve (for example, AW300) was suspended in dried CH₂Cl₂ (4.0 mL). The mixture was stirred at the room temperature for 10 minutes, and then stirred at −10° C. for 10 minutes. Then, in the absence of an acceptor, NIS (77 mg, 0.34 mmol) and TMSOTf (54 μL, 0.3 mmol) were added. After the activation was performed at −10° C. for 1.5 hours, the thioglucoside acceptor (17 mg, 0.2 mmol, according to C.-S. Chao, Y.-F. Yen, W.-C. Hung, K.-K. T. Mong, Adv. Synth. Catal. 2011, 353, 879-884.) was added, and the addition reaction was performed at −10° C. for 3 hours. After the addition reaction, saturated NaHCO₃ and sodium sulfite were added. Then, the mixture was dried with magnesium sulfate, filtered and analyzed by chromatography (hexane/EtOAc/CH₂Cl₂: 6/1/3), so as to obtain the white glass compound (172 mg, 85%, α/β=49:1).

α-isomer had ¹H NMR (300 MHz, CDCl₃): δ 7.96 (d, J=7.2 Hz, 2H, ArH), 7.79 (d, J=7.5, 2H, ArH), 7.57-7.53 (m, 2H, ArH), 7.51-7.45 (m, 2H, ArH), 7.43-7.21 (m, 19H, ArH), 7.10-7.04 (m, 7H, ArH), 5.69 (t, J=9.3, 1H), 5.48 (s, 1H, benzylidene-CH), 5.33 (t, J=9.6 Hz, 2H), 5.17 (d, J=3.3 Hz, 1H, H-1), 4.87-4.73 (m, 4H), 4.65 (d, J=11.7 Hz, 1H), 4.49 (s, 2H), 4.22 (d, J=12.3, 1H), 4.11-4.07 (m, 2H), 4.01-3.83 (m, 5H), 3.79-3.74 (m, 1H), 3.65 (s, 1H), 2.23 (s, 1H, CH₃); ¹³C NMR (75 MHz, CDCl₃): δ 166.1 (C═O), 165.7 (C═O), 139.2, 139.0, 138.7, 138.4, 137.8, 133.6, 133.5, 130.32, 130.25, 129.84, 129.76, 129.4, 128.93, 128.78, 128.7, 128.6, 128.5, 128.4, 128.2, 128.1, 128.04, 128.00, 126.8, 101.5 (benzylidene-CH), 98.5 (C-1′), 86.2 (C-1), 80.0, 77.9, 77.5, 77.1, 76.8, 76.5, 76.4, 76.0, 75.1, 74.0, 72.4, 71.3, 69.9, 66.1, 63.1, 21.6 (CH₃); HRMS (m/z): [M+Na]⁺ C₆₁H₅₈NaO₁₂S calculated as 1037.3541; measured as 1037.3493.

### Embodiment 4

### Synthesis of 10-chlorodecanyl 2,3-di-O-benzyl-4,6-O-benzylidene-α-D-galactopyranoside

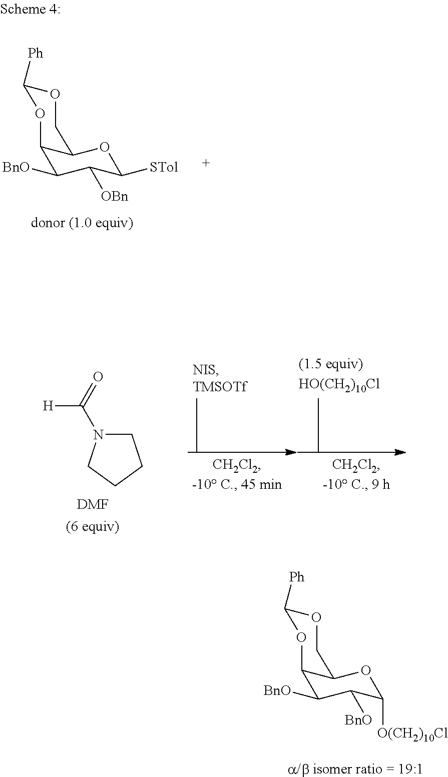

The compound of Embodiment 4 was synthesized according to Scheme 4.

The mixture of a donor having thiogalactoside (166.3 mg, 0.3 mmol), N-formylpyrrolidine (110 μL, 1.2 mmol) and a flame-dried molecular sieve (for example, AW300) was suspended in dried CH₂Cl₂ (4.0 mL). The mixture was stirred at the room temperature for 10 minutes, and then stirred at −10° C. for 10 minutes. Then, in the absence of an acceptor, NIS (77 mg, 0.34 mmol) and TMSOTf (54 μL, 0.3 mmol) were added. After the activation was performed at −10° C. for 45 minutes, the 1-chlorodecanol acceptor (86 mg, 0.45 mmol in 1 mL of CH₂Cl₂) was added, and the addition reaction was performed at −10° C. for 3 hours. After the addition reaction, saturated NaHCO₃ and sodium sulfite were added. Then, the mixture was dried with magnesium sulfate, filtered and analyzed by chromatography (hexane/EtOAc/CH₂Cl₂: 7/0.5/2), so as to obtain the white glass compound (133 mg, 72%, α/β=19:1).

α-isomer had ¹H NMR (300 MHz, CDCl₃): δ 7.53-7.50 (m, 2H, ArH), 7.42-7.25 (m, 13H, ArH), 5.46 (s, 1H, benzylidene-C H), 4.91 (d, J=3.3 Hz, 1H, H-1), 4.86 (d, J=10.2 Hz, 1H), 4.82 (d, J=10.2 Hz, 1H), 4.75-4.64 (m, 2H), 4.20 (dd, J=1.3, 12.3 Hz, 1H), 4.19 (d, J=3 Hz, 1H), 4.10-3.67 (m, 3H), 3.65-3.59 (m, 2H), 3.5 (t, J=6.6 Hz, 2H, CH₂), 3.44 (m, 1H), 1.8-1.7 (m, 2H, CH₂), 1.6-1.5 (m, 2H, CH₂), 1.44-1.37 (m, 2H, CH₂), 1.28 (broad, 10H, CH₂×₅); ¹³C NMR (75 MHz, CDCl₃): δ 138.9, 138.8, 137.8, 128.8, 128.2, 128.0, 127.8, 127.57, 127.52, 127.40, 127.36, 126.3, 101.1 (benzylidene-CH), 98.0 (C-1), 76.1, 75.8, 75.3, 74.8, 73.4, 72.1, 69.3, 68.4, 62.6 (CH₂O), 45.1 (CH₂Cl), 32.6 (CH₂), 29.4 (CH₂), 29.3 (CH₂), 28.8 (CH₂), 26.8 (CH₂), 26.1 (CH₂).

### Embodiment 5

### Synthesis of methyl 2-azido-3,4,6-tri-O-benzyl-2-deoxy-D-glucopyranosyl-α(1,4)-2,3-O-isopropylidene-1-α-L-rhamnopyranoside

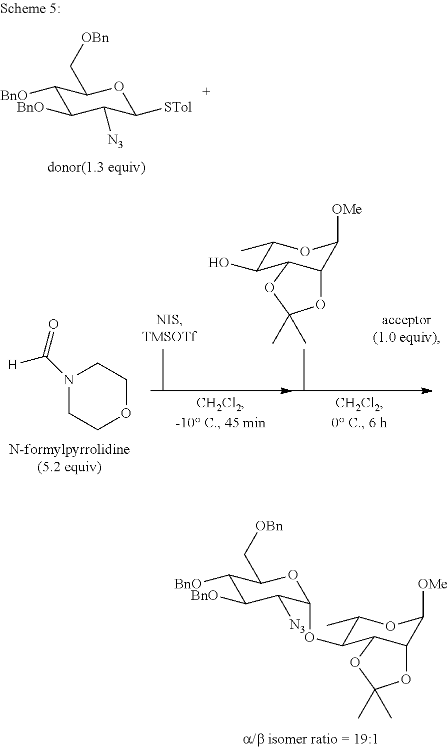

The compound of Embodiment 5 was synthesized according to Scheme 5.

The mixture of a donor having thiogalactoside (173.2 mg, 0.3 mmol, according to C.-S. Chao, C.-W. Li, M.-C. Chen, S.-S. Chang, K-K. T. Mong, Chem. Eur. J. 2009, 15, 10972-10982), N-formylmorpholine (125 μL, 1.2 mmol) and a flame-dried molecular sieve (for example, AW300) was suspended in dried CH₂Cl₂ (4.0 mL). The mixture was stirred at the room temperature for 10 minutes, and then stirred at −10° C. for 10 minutes. Then, in the absence of an acceptor, NIS (77 mg, 0.34 mmol) and TMSOTf (54 μL, 0.3 mmol) were added. After the activation was performed at −10° C. for 45 minutes, the rhamnoside acceptor (50 mg, 0.23 mmol in 2 mL of CH₂Cl₂) was added, and the addition reaction was performed at 0° C. for 6 hours. After the addition reaction, saturated NaHCO₃ and sodium sulfite were added. Then, the mixture was dried with magnesium sulfate, filtered and analyzed by chromatography (hexane/EtOAc/CH₂Cl₂: 6/1/2), so as to obtain the white glass compound (89 mg, 60%, α/β=19:1).

α-isomer had ¹H NMR (300 MHz, CDCl₃): δ 7.41-7.31 (m, 13H, ArH), 7.25-7.23 (m, 2H, ArH), 5.07 (d, J=3.6 Hz, 1H, H-1), 4.94-4.85 (m, 4H), 4.67 (d, J=12 Hz, 1H), 4.62 (d, J=10.8 Hz, 1H), 4.55 (d, J=12 Hz, 1H), 4.16-4.11 (m, 3H), 4.02 (t, J=9 Hz, 1H), 3.93-3.85 (m, 2H), 3.78-3.67 (m, 2H), 3.47 (dd, J=3.9, 10.2 Hz, 1H), 3.39 (s, 3H, OCH₃), 1.48 (s, 3H, CH₃), 1.42 (d, 3H, CH₃), 1.31 (s, 3H, CH₃); ¹³C NMR (75 MHz, CDCl₃): δ 138.5, 138.34, 130.30, 128.92, 128.89, 128.83, 128.5, 128.36, 128.28, 128.24, 128.17, 128.04, 109.5 (benzylidene-C), 99.0 (C-1), 98.2 (C-1′), 81.4, 80.7, 78.6, 76.3, 75.8, 75.5, 74.0, 3.9, 71.1, 68.2 (CHN₃), 28.6 (CH₃), 26.8 (CH₃), 17.9 (CH₃).

### Embodiment 6

### α-Glycosylation Reaction in One Pot Environment

### Synthesis of methyl 2,3,4,6-tetra-O-benzyl-D-galactopyranosyl-α(1,3)-2,4,6-tri-O-benzyl-D-glucopyranosyl-α(1,3)-2,4-di-O-benzyl-L-rhamnopyranoside

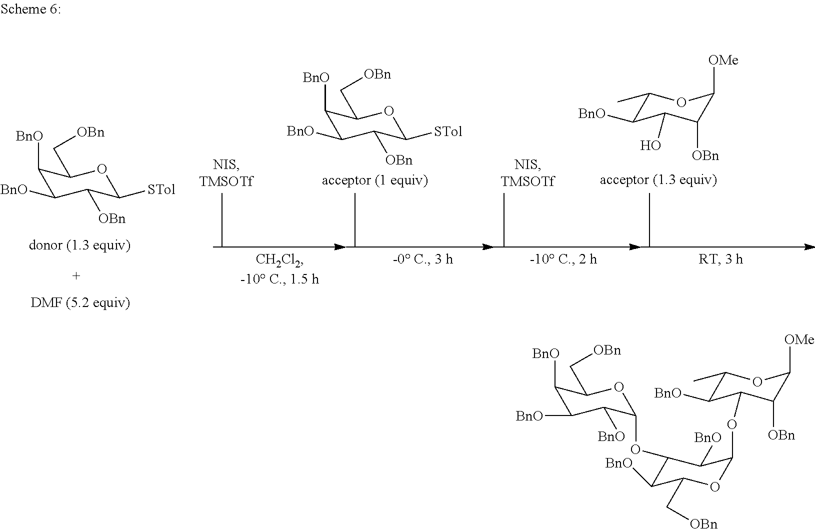

The compound of Embodiment 6 was synthesized according to Scheme 6.

The mixture of a donor having thiogalactoside (65 mg, 0.1 mmol, according to Z. Zhang, I. R. Ollmann, X.-S. Ye, R. Wischnat, T. Baasov, C.-H. Wong, J. Am. Chem. Soc. 1999, 121, 734-753), DMF (31 μL, 0.4 mmol) and a flame-dried molecular sieve (for example, AW300) was suspended in dried CH₂Cl₂ (2.0 mL). The mixture was stirred at the room temperature for 10 minutes, and then stirred at −10° C. for 10 minutes. Then, in the absence of an acceptor, NIS (23 mg, 0.1 mmol) and TMSOTf (19.5 μL, 0.1 mmol) were added. After the activation was performed at −10° C. for 1.5 hours, the thiglucoside acceptor (43 mg, 0.077 mmol) was added, and the addition reaction was performed at 0° C. for 3 hours. Then, the mixture was stirred for 10 minutes, and cooled on ice to −10° C. In the presence of DMF, NIS (18 mg, 0.079 mmol) and TMSOTf (23 μL, 0.13 mmol) were added. After the reaction was performed for 2 hours, the rhamnoside acceptor (36 mg, 0.1 mmol) was added, and the reaction was performed at 20° C. for 3 hours. As previously illustrated, the mixture was then dried, and analyzed by chromatography (hexane/EtOAc/CH₂Cl₂=5/1/1), so as to obtain the yellow white compound (55 mg, 42%, single stereoisomer).

¹H NMR (500 MHz, CDCl₃): δ 7.34-7.09 (m, 45H, ArH), 7.05 (t, J=7.5 Hz, 2H, ArH), 6.94 (dd, J=1.5, 7.8 Hz, 2H, ArH), 5.63 (d, J=3.5 Hz, 1H, H-1″), 5.22 (d, J=3.5 Hz, 1H, H-1′), 4.88-4.78 (m, 4H), 4.74-4.63 (m, 6H, [symbol] H-1), 4.62-4.53 (m, 4H), 4.51-4.44 (m, 3H), 4.36-4.28 (m, 3H), 4.22 (d, J=2.5 Hz, 3H), 4.08-4.02 (m, 2H), 3.96 (dd, J=2.5, 10.3 Hz, 2H), 3.89-3.82 (m, 3H), 3.69 (dd, J=3.0, 10.0 Hz, 1H), 3.66-3.61 (m, 1H), 3.59-3.55 (m, 1H), 3.50-3.43 (m, 2H), 3.40-3.38 (m, 2H); ¹³C NMR (125 MHz, CDCl₃): δ=139.24, 139.16, 138.9, 138.8, 138.7, 138.5, 138.4, 138.2, 128.9, 128.80, 128.79, 128.75, 128.74, 128.72, 128.63, 128.62, 128.59, 128.57, 128.4, 128.2, 128.1, 128.02, 127.97, 127.94, 127.92, 127.89, 127.83, 127.6, 127.3, 99.4 (C-1), 98.0 (C-1″), 94.1 (C-1′), 80.2, 79.7, 79.5, 79.1, 76.1, 75.92, 75.86, 75.7, 75.6, 75.3, 75.0, 74.3, 73.9, 73.74, 73.65, 73.58, 73.4, 73.0, 70.7, 69.11, 69.06, 68.7, 68.6, 55.1, 30.2, 18.4. HRMS (MALDI-TOF): C₈₂H₈₈O₁₅Na [M+Na]⁺ calculated as 1335.6021; measured m/z=1335.6015.

In the method of the present invention, the reaction of the donor having a saccharide structure and the formamide-containing compound is performed to form a glycosyl imidate compound, and then the coupling reaction of the imidate compound with an acceptor having a hydroxyl group is performed in the one pot environment to give the α-glycoside. In the present invention, α-glycoside with high to excellent selectivity of glycosylation (α/β ratio 19:1 to 49:1) is achieved. Accordingly, the method of the present invention is simple, suitable for the large scale production, and easy to recover the formamide-containing compound.

The foregoing descriptions of the detailed embodiments are only illustrated to disclose the features and functions of the present invention and not restrictive of the scope of the present invention. Accordingly, the protection sought herein is as set forth in the claims below.

What is claimed is:

1. An α-selective glycosylation method, comprising the steps of:
performing a reaction of a donor having a saccharide structure and a formamide-containing compound to form a glycosyl imidate compound; and
performing a coupling reaction of the glycosyl imidate compound with an acceptor having a hydroxyl group to form an α-glycoside.

2. The α-selective glycosylation method of claim 1, wherein a carbon atom at the first position of the saccharide structure has a substitute, and the substitute is activated by an activating agent.

3. The α-selective glycosylation method of claim 2, wherein the carbon atom at the first position of the saccharide structure is substituted with a thioacetal group, and the activating agent is a halonium ion source.

4. The α-selective glycosylation method of claim 2, wherein the halonium ion source is a mixture of N-halosuccinimide and a Lewis acid.

5. The α-selective glycosylation method of claim 4, wherein the N-halosuccinimide is iodosuccinimide or bromosuccinimide, and the Lewis acid is triflic acid, trimethylsilyl triflate or silver triflate.

6. The α-selective glycosylation method of claim 1, wherein a carbon atom oat the first position of the saccharide structure is substituted with a thioacetal, a halo, a phosphate or an acetimidate.

7. The α-selective glycosylation method of claim 6, wherein the thioacetal is thiotoluenyl acetal or thiophenyl acetal.

8. The α-selective glycosylation method of claim 6, wherein the acetimidate is trichloroacetimidate or N-phenyl trifluoroacetimidate.

9. The α-selective glycosylation method of claim 6, wherein the phosphate is diphenyl phosphate.

10. The α-selective glycosylation method of claim 6, wherein the saccharide structure has an active atom with a protecting group, and the active atom is O or N.

11. The α-selective glycosylation method of claim 1, wherein the saccharide structure has 6 or more carbon atoms.

12. The α-selective glycosylation method of claim 11, wherein the saccharide structure is a linear or circular structure.

13. The α-selective glycosylation method of claim 1, wherein the donor is D-galactose, D-glucose, 2-azido-2-deoxy-D-galactose, 2-azido-2-deoxy-D-glucose, L-idopyranose, D-mannose, or L-rhamnose.

14. The α-selective glycosylation method of claim 1, wherein the formamide-containing compound has a structure of formula (I): 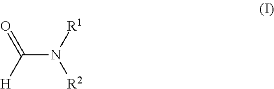
wherein R¹ and R² are independently C₁-C₆alkyl or R¹ and R² are part of a 5-membered or 6-membered heterocyclic compound that bears one or more heteroatoms.

15. The α-selective glycosylation method of claim 14, wherein the heterocyclic compound has at least one carbon atom replaced by an oxygen atom.

16. The α-selective glycosylation method of claim 14, wherein the formamide-containing compound is N,N-dimethylformamide, N,N-diethylformamide, N,N-diisopropylformamide, N-formylpyrrolidine, N-formylpiperidine or N-formylmorpholine.

17. The α-selective glycosylation method of claim 1, further comprising the steps of:
activating the α-glycoside to react with the formamide-containing compound; and
performing a coupling reaction with the acceptor having the hydroxyl group.
